(19) United States
(12) Patent Application Publication
(10) Pub. No.: US 2024/0027429 A1
(43) Pub. Date: Jan. 25, 2024

(54) Time Dependent Response of Basophils to Allergens

(71) Applicants: Oral Alpan, Fairfax, VA (US); Soren Sonder, Fairfax, VA (US)

(72) Inventors: Oral Alpan, Fairfax, VA (US); Soren Sonder, Fairfax, VA (US)

(21) Appl. No.: 18/213,417

(22) Filed: Jun. 23, 2023

Related U.S. Application Data
(60) Provisional application No. 63/355,828, filed on Jun. 27, 2022.

(51) Int. Cl. G01N 33/50; G01N 1/30; G01N 33/68

(52) U.S. Cl. CPC G01N 33/5094; G01N 1/30; G01N 33/6872; G01N 2333/70596

(57) ABSTRACT

Food allergens and environmental allergens are more readily assessed in vitro than with cumbersome and variable skin tests. Methods for measuring basophil response to allergens are improved when a variety of time points are interrogated and rates of change considered. Interrogation of internal and external markers of basophil activation also improves assessments.

### TECHNICAL FIELD OF THE INVENTION

This invention relates to diagnosing and monitoring food allergies with basophil activation tests. The assessment can be performed on unstimulated and stimulated basophils over a time gradient.

### BACKGROUND OF THE INVENTION

Basophil activation testing was first described in 1993. Incubation of whole blood samples with substances to which the patient is allergic to (e.g., peanut), resulted in increases of the appearance of some of the basophil surface molecules such as CD203c and CD63. The test had limited clinical utility due to the fact that basophils of 10-20% of patients are non-reactive or poorly reactive. This has been shown to be due to the lack of an intracellular signaling molecule called Syk in non-responder subjects. Additionally, the clinical utility is limited by the instability of whole blood samples beyond 6 hours, because in most cases samples are shipped to a central laboratory for analysis.

The intensity of responses of basophils of patients to incubation (stimulation) with an actual allergen are different than with a positive control (i.e., anti-IgE or fMLP). This is seen in the differential staining intensity of CD63 and CD203c when measured by flow cytometry. Such staining intensity may correlate with the severity of the clinical reactivity, however, this has not been clearly established in the medical literature.

Basophils account for approximately 0-2% of white blood cells. Basophils play important roles in Immunoglobulin E-mediated (IgE-mediated) immune responses including food allergies, asthma, and environmental allergens. The basophil count often increases prior to the onset of symptoms and the activation state of basophils may correlate with allergic disease symptoms.

Food allergy is a major public health issue that affects as many as 3-4% of adults and 8% of children in the United States. The incidence of allergic diseases and food allergies has increased significantly in developed countries in the last three decades. Currently used, first-line methods for identifying offending antigens are based on in vivo as well as in vitro allergen tests.

The in vivo allergen testing is usually carried out as a skin test, which at times can be inconclusive. In vitro allergen tests include measurement of allergen specific IgE levels by various tests. These assays are blood-based, and have low specificity and sensitivity, partly due to the fact that they are static tests as opposed to functional tests.

The gold standard in diagnosis of food allergies is an oral food challenge which involves the gradual administration of the food allergen over a short period of time (hours) and observing the patient for a reaction. This test is not only difficult to perform, it is also time-consuming and, potentially dangerous since it can result in anaphylactic reactions and even death, if treatment is not initiated quickly. Such tests create significant anxiety among patients. Hence the in vitro basophil activation test is an attractive alternative to replace the oral food challenge test. The strong negative predictive value of the basophil activation test prevents patients from continuing to unnecessarily avoid foods and environments, and avoids unnecessary treatments (e.g., oral food immunotherapy or epinephrine usage).

### SUMMARY OF THE INVENTION

One aspect of the invention is a method of preparing samples to aid in determining likelihood of a subject having an allergic reaction against an allergen of interest. Basophil cells of the subject in a first blood sample are stimulated in vitro by contacting the basophil cells with a selected concentration of the allergen of interest for a first period of time. Basophil cells of the subject in a second blood sample are stimulated in vitro by contacting the basophil cells with a selected concentration of the allergen of interest for a second period of time. The first and second blood samples are then subjected to an analysis of basophil cell activation. The rate of change between the first and second time period endpoints is determined.

Another aspect of the invention is a method of treating a subject who is prone to having an allergic reaction against an allergen of interest. Basophil cells of the subject in a first blood sample are stimulated in vitro by contacting the basophil cells with a selected concentration of the allergen of interest for a first period of time. Basophil cells of the subject in a second blood sample are stimulated in vitro by contacting the basophil cells with a selected concentration of the allergen of interest for a second period of time. The first and second blood samples are then subjected to an analysis of basophil cell activation. The rate of change between the first and second time period endpoints is determined. The subject is treated with desensitizing immunotherapy toward the allergen of interest when the rate is determined to be significantly above negative control values.

Another aspect of the invention is a set of assay samples to aid in determining likelihood of a subject having an allergic reaction against an allergen of interest. The set comprises a plurality of assay samples comprising at least a first and a second blood sample. The first and second blood samples are formed by stimulating in vitro basophil cells in a first blood sample of the subject by contacting the basophil cells with a selected concentration of the allergen of interest for a first period of time and by stimulating basophil cells in vitro in a second blood sample of the subject by contacting the basophil cells with the selected concentration of the allergen of interest for a second period of time. The plurality of assay samples are suitable for measuring one or more markers of basophil activation in the blood samples whereby a rate of basophil activation can be determined by determining a change in amount of the one or more markers between any of the blood samples.

A further aspect of the invention is a kit to aid in determining likelihood of a subject having an allergic reaction against an allergen of interest. The kit may comprise a plurality of tubes for holding at least a first and a second blood sample of the subject in which basophil cells will be stimulated by contacting the basophil cells with a selected concentration of the allergen of interest. The kit may comprise the allergen of interest at a predetermined amount in separate packages to be added to the at least first and second blood samples. And the kit may comprise a quenching agent for stopping the stimulation of basophil cells at a predetermined amount in separate packages to be added to the at least first and second blood samples.

An additional aspect of the invention is a method to aid in determining likelihood of a subject having an allergic reaction against an allergen of interest. Basophil cells of the subject in a first blood sample are stimulated in vitro by contacting the basophil cells with a selected concentration of the allergen of interest for a first period of time. Basophil cells of the subject in a second blood sample are stimulated in vitro by contacting the basophil cells with a selected concentration of the allergen of interest for a second period of time. The first and second blood samples are then subjected to an analysis of basophil cell activation by measuring one or more markers of basophil activation. The rate of change of marker expression between the first and second time period endpoints is determined to determine a rate of basophil activation.

These and other embodiments which will be apparent to those of skill in the art upon reading the specification provide the art with

### BRIEF DESCRIPTION OF THE DRAWINGS

FIG. 1: Basophil identification: Gating strategy. Initially a forward scatter (FCS)/side scatter (SSC), singlet (FCS-A/FCS-H) and CD45/SSC are applied. This is followed by gating on basophils using both a CD123+/CD193+ and an IgE+/SSClow gate. Basophil activation is determined by measuring percentages of CD63 positive basophils (especially CD63—bright staining) as well as the fold change in CD203c MFI compared to the negative control.

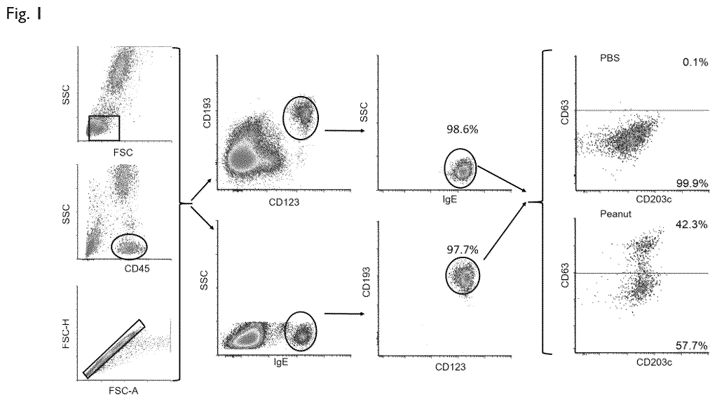

FIG. 2A-2B. Levels of CD203c (FIG. 2A) and CD63 (FIG. 2B) surface expression on basophils measured at 10, 20, 40 and 60 minute timepoints after ex vivo peanut stimulation with varying allergen concentrations showed a time-response curve that correlated with the severity of the clinical allergic reactions to the oral food challenge. Those who did not have a severe reaction on oral challenge only had a slow rate of CD63 up-regulation over a time course tested. Similar pattern of time-response curves was observed with phospho-proteins stained (pLyn, pZap-70 and others).

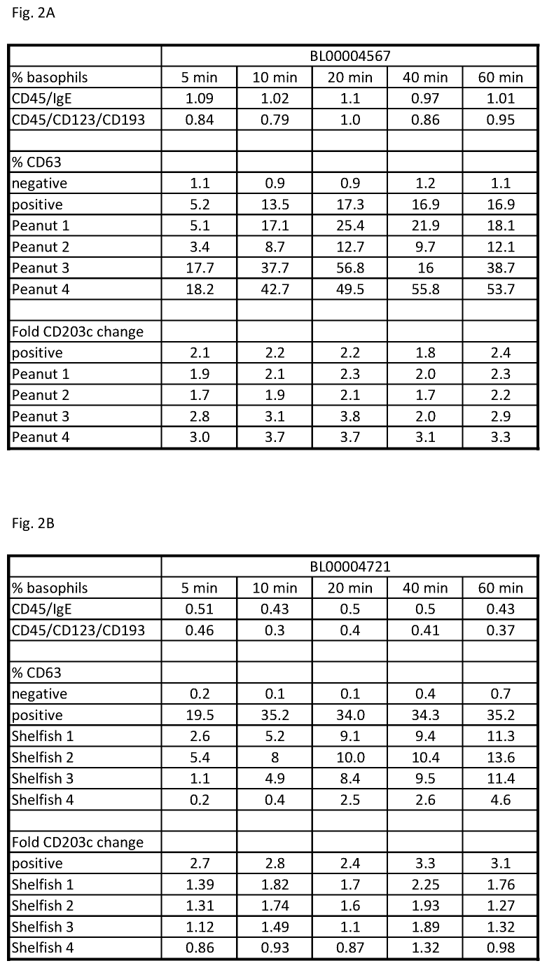

FIG. 3A-3D. The staining shows phosphorylation of basophil phosphoproteins. Cytoplasmic expression of pPLCg1 (Tyr783) and pZAP (Tyr319)/SYK (354) in Basophils activated with either PBS or IgE as shown. Whole blood was incubated with either PBS or IgE for 30 minutes at 37 degrees Celsius. Activated Sodium Orthovanadate (5 mM) was added for 15 minutes during stimulation (FIG. 3B & FIG. 3D). Whole blood was incubated with Basophil identification antibodies (IgE, CD123, CD193, CD45, CD203c) for 30 minutes at 4 degrees Celsius. The whole blood was lysed with BD Facs Lyse, washed, then permeabilized with BD Cytofix/Cytoperm. Cells were then incubated with the anti-phospho antibodies (FIG. 3A & FIG. 3B) pPLCg1 (Tyr783) or (FIG. 3C & FIG. 3D) pZAP (Tyr319)/SYK (354) for 60 minutes at 4 degrees Celsius. Samples were acquired on an Attune NxT Flow Cytometer (ThermoFisher Scientific).

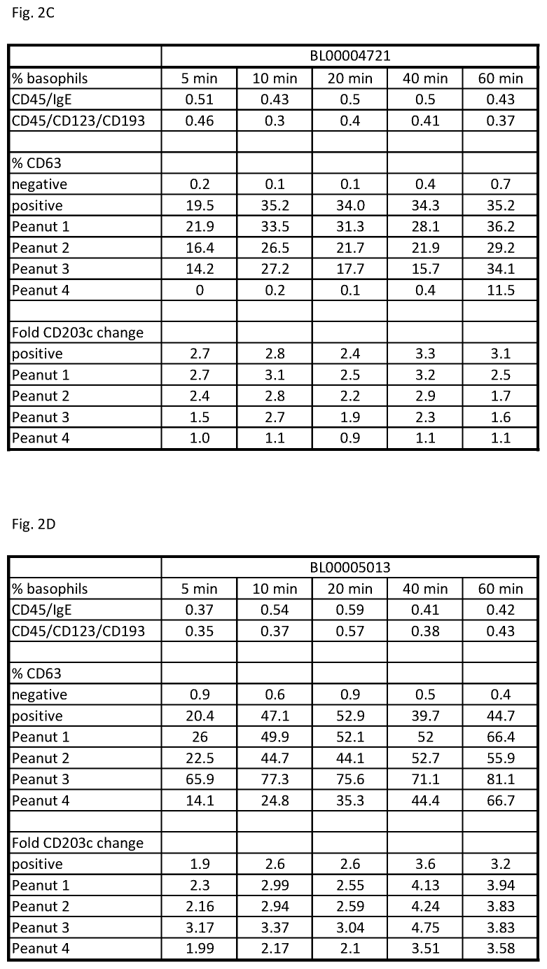

FIG. 4. Basophil activation through the IgE pathway can be inhibited by the btk inhibitor Ibrutinip. This allows one to assess the non-specific (i.e., non-IgE dependent) stimulation of basophils, as fMLP stimulation of basophils is not effected by btk inhibitor ibrutinub and other btk inhibitors at concentration above 0.01 micromolar.

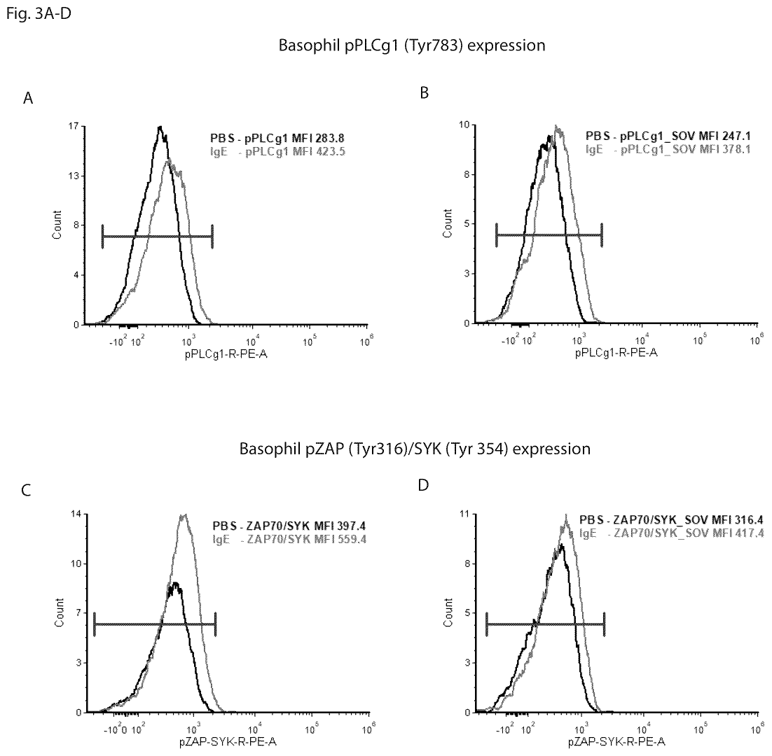

### DETAILED DESCRIPTION OF THE INVENTION

The present invention addresses basophil activation tests performed in a way in which basophil surface and/or intra-cellular phosphorylation of signaling molecules are measured, for example, over 20 minutes at intervals of seconds up to several minutes (e.g., 1, 3, 5, 10 and 20 minutes or all other combinations of intervals up to about 60 minutes after stimulation). The method is based on the detection of basophil surface and/or intracellular phosphorylation proteins, following stimulation by the allergen of interest. The allergen may be at concentrations between 0.1 to 100,000 ng/ml, depending on the allergen used. The surface and intra-cellular markers may be conveniently measured by flow cytometry, when, for example, fluorescent labeled antibodies are used as detectors for the basophil activation markers. Other methods for measuring activation markers or their expression may be used.

In vivo determination of a subject's susceptibility to an allergic reaction upon exposure to an allergen of interest may be used, for example, to compare to the in vitro methods disclosed here. The subject may have a suspected allergy or may have no known allergy to the allergen or known predisposition. This should requires trained medical personnel because of the risk of life-threatening reactions and the need for emergency interventions.

Whole blood may be collected from the subject, and incubated with the allergen for varying durations. In most cases a single concentration will be used, but in some cases it may be desirable to use various concentrations. Blood cells may then be analyzed for the presence, amount, or expression of cell surface and intracellular markers indicative of basophil activation and/or an allergic response. Exemplary markers include CD203c, CD63, MRGPRX2, phopsho-Lyn, phospho-Fyn, phospho S6, phospho AKT and phospho-IgE receptor, as well as other intracellular phosphorylation markers. The phosphorylated targets IgE receptor, Fyn, and Lyn are proximal to (upstream in the pathway from) Syk and are unaffected by the absence of Syk in non-responder patients. Thus this method allows successful testing in those patients with non-reactive basophils based on conventional cell surface markers (e.g., CD63 or CD203c).

The methods of the invention may be used in the diagnosis or monitoring of allergies in an individual, or monitoring of effectiveness of a therapeutic modality with respect to the responsiveness of an individual being treated. The methods may also be used in a research and screening context for identifying and developing new therapeutic agents for treating allergic diseases and other conditions which involve basophil cell activation.

The disclosed method addresses the problem of non-releaser or low-responding basophils that is seen in 10-20% of the population. The measurement of the rate of up-regulation of these molecules over a short time period increases the sensitivity and the specificity of classic basophil activation testing to determine clinical reactivity.

In another embodiment, an inhibitor of basophil activation (btk inhibitor ibrutinib) is used. Btk inhibitors inhibit the activation of the IgE stimulation pathway. This allows one to assess if basophil activation induced by the allergen is specific, as the inhibitor will not affect fMLP (N-Formylmethionyl-leucyl-phenylalanine) induced basophil activation, but only the IgE pathway. fMLP is a positive control used to assess if the basophils respond to a non-IgE pathway stimulation, hence measuring overall basophil reactivity.

Methods of the invention provide an in vitro, whole blood based method of determining a subject's likelihood to experience an allergic reaction upon exposure to an allergen of interest. The method is based on the monitoring and detection of cell surface and/or intracellular molecules on/in basophil cells from the subject. The cells are stimulated in vitro by the allergen over a plurality of time periods. The activation markers are measured and analyzed and a rate of up-regulation can be determined. If desired, a dose-response curve can be generated. If desired, a time course of activation can be generated.

Particular embodiments of the invention determine basophil response to the binding of allergen to allergen-specific IgE or other relevant Ig in whole blood, without prior isolation of the basophils. There is no need to pre-isolate the basophils, since their activation may be detected by flow cytometric assays in the midst of the other cells in the blood sample. Flow cytometry permits functional isolation and detection of specific signaling in basophils.

According to one aspect, the present invention provides a method for predicting a subject's response to an allergen challenge by testing a whole blood sample obtained from a subject suspected of an allergy. The method comprises the steps of contacting the blood sample with a selected concentration of an allergen over a number of time points and assessing the level of expression of a cell surface and/or an interior marker that is characteristic of activation of basophil cells. The expression of the marker in the blood sample correlates with the subject's response to allergen challenge. The expression may be compared, for example, to a control sample in which allergen is not present. Multiple markers may be assessed, if desired.

The blood sample that provides a source of basophils may be obtained by any convenient method. Whole blood may be used. An anti-clotting agent may be added to the sample, such as heparin. Other anticoagulants have been observed not to work as well for this test. Blood is preferably used within about 48 hours from collection from the subject.

The candidate allergen may be added to the blood sample, at a concentration of 0.1 to 100,000 ng/mL. Preferred allergen extracts are standardized and are commercially available. A positive control may also be used, for example, using polyclonal IgE at 1 μg/ml. The cells may be incubated with the allergen for a plurality of periods of time so that basophil activation can be observed and measured. The time points may be measured in seconds to minutes, preferably up to 20 minutes, but typically not more than about 1 hour.

The sample may be contacted with binding reagents that selectively bind to (a) a marker for basophil activation; and optionally (b) a marker that distinguishes basophils from other blood cells. Antibodies are a preferred reagent and may be directly or indirectly labeled. Useful markers for basophil activation include without limitation one or more of CD203c, CD63, Ph-Lyn, Ph-Fyn, Ph-PLC, Ph-ZAP70 and other intracellular, phosphorylation related markers. In preferred embodiments the marker for basophil activation is one or both of CD203c and CD63.

Markers that can be used to distinguish basophils from other blood cells include, CD123, CD193, IgE, Fad HLA-DR (negative), CRTH2 and CD3 (negative) and the like. Antibodies specific for these markers can be used to detect and/or measure them.

Calculation of the Slope of the Curve:

The slope of the curve of a basophil activation test when measured over time as described in the summary of the invention is non-linear.

One of the differences between the slope of a straight line and the slope of a curve is that the slope of a straight line is constant, while the slope of a curve changes from point to point.

To find the slope of a line you need to:
1. Step One: Identify two points on the line.
2. Step Two: Select one to be (x1, y1) and the other to be (x2, y2).
3. Step Three: Use the slope equation to calculate slope.

$$slope = \frac{( {y}_{2} - {y}_{1} )}{( {x}_{2} - {x}_{1} )}$$

Method A: To use the slope formula in a nonlinear relationship:

Here we see that the slope of the curve changes as we move along it. For this reason, we measure the slope of a curve at just one point. For example, instead of measuring the slope as the change between any two points (between A and B or B and C), we measure the slope of the curve at a single point (at A or C). Each of the single points represent a time point of the stimulation as described in the summary of the invention.

To do this we introduce the concept of a tangent (Method B). A tangent is a straight line that touches a curve at a single point and does not cross through it. The point where the curve and the tangent meet is called the point of tangency. Both of the figures below show a tangent line to the curve.

The slope of a curve at a point is equal to the slope of the straight line that is tangent to the curve at that point.

### Example

What is the slope of the curve at point A?

This is the slope of the curve only at point A. To find the slope of the curve at any other point, we would need to draw a tangent line at that point and then determine the slope of that tangent line.

In the basophil activation test, the slope of the curve may be determined for both the positive control anti-IgE as well as the allergen (e.g., peanut) using Method A and/or Method B. A slope equivalent or higher than to that of the positive control, may be used to indicate clinical allergy.

The term “subject,” “mammalian subject,” “individual,” or “patient” may be used interchangeably to refer to a member of a species of mammalian origin, including but not limited to a human, mouse, rat, cat, goat, sheep, horse, hamster, ferret, pig, dog, guinea pig, rabbit or primate, adult or not yet adult.

The terms “allergic response” and “allergy” may be used interchangeably to describe an abnormal reaction of the body to a previously encountered allergen introduced by inhalation, ingestion or skin contact. The use of these terms also includes clinically adverse reactions to environmental allergens which reflect the expression of acquired immunologic responsiveness involving allergen-specific antibodies and/or T cells. These terms also include adverse immunologic responses that are associated with the production of allergen-specific IgE.

The term “allergen” may refer to any substance that induces an allergy in a susceptible subject. The use of the term “allergen” includes any antigens that elicit a specific IgE response. Allergens may have little or no intrinsic toxicity by themselves, but cause a pathological condition due to their ability to elicit an IgE-associated immune response, and, upon subsequent exposure, due to their ability to elicit IgE- and/or T cell-dependent hypersensitivity reactions. Common allergens include but are not limited to pollen, grasses, dust, as well as foods, including, but not limited to, nuts, milk, eggs, shell fish, and venoms, and various drugs. Allergens include, without limitation, nanoparticles, metal or metal alloys, drug or medicine related antigens; various biological matters, e.g., proteins, which may be related to animals such as insects or arachnids. Other allergens may be related to humidifiers and air conditioners.

The term “allergic diseases” may be used to reference a group of clinically manifested disorders in which immune responses, typically directed against otherwise innocuous environmental allergens, are thought to have a pathogenetic role. Allergic diseases include, but are not limited to, hay fever, allergic asthma, allergic contact dermatitis, and clinical disorders in which IgE-associated immune responses are thought to play a role.

The term “activation” may refer to a physiological condition upon exposure to a substance, allergen, drug, protein, chemical, or other stimulus, or upon removal of a substance, allergen, drug, protein, chemical or other stimulus.

Positive control antigen may be anti-IgE, anti-IgG, anti-IgD antibodies, fMLP, cytokines, IL-3, IL-18, IL-33, histamine, anti-Fc receptor antibodies, PMA/Ionomycin, PMA/cal, proteases enzymes, papain, TLR receptor/ligand or antibodies against TLR or agonists, complement factors, antigens from helmiths, ROS pathway markers, or other intracellular or extracellular markers that are involved in the basophil activation or degranulation.

The term “activation marker” may refer to cell surface markers indicative of basophil activation, for example one or both of CD203c and CD63; CD13, CD107a, CD164, CD80, CD86, CD40L, HLA-DR, CD123, CRTH2 and other extracellular markers on basophils, or intracellular markers such as Ph-CREB, Ph-STATS, Ph-S6rp, Ph-eIF4E, CREB or mTOR pathway proteins, or other phosphorylation related markers, or other proteins or small molecules related to the activation of basophils. CD203c and CD63 are of particular interest. The physiological condition may be the result of an exposure to a substance, allergen, drug, protein, chemical, or other stimulus, or maybe the result of removal of a substance, allergen, drug, protein, chemical or other stimulus.

The term “cell surface marker” may refer to an antigenic determinant or epitope found on the surface of a specific type of cell. Cell surface markers can facilitate the characterization of a cell type, its identification, and eventually its isolation. Cell sorting techniques are based on cellular biomarkers where one or more cell surface markers are used for either positive or negative selection, i.e., for inclusion or exclusion, from a cell population.

The term “cytometry” may refer to a process in which physical and/or chemical characteristics of single cells, or by extension, of other biological or nonbiological particles in roughly the same size or stage, are measured. In flow cytometry, the measurements are made as the cells or particles pass through the measuring apparatus (flow cytometer) in a fluid stream. A cell sorter, or flow sorter, is a flow cytometer that uses electrical and/or mechanical means to divert and collect cells (or other small particles) with measured characteristics that fall within a user-selected range of values.

A marker that distinguishes basophils from other blood cells may refer to a detectable physical parameter, particularly a parameter that can be monitored by flow cytometry, hat allows basophils to be “gated” or separately analyzed from other blood cells. Markers of particular interest include size (detectable by forward scatter), granularity (detectable by side scatter) and cell surface markers, which can be detected by antibody staining for the marker of interest. In some embodiments of the invention, a cell sample is stained with one or more antibodies that distinguish between basophils and T cells, such as CCR3, CD3, CD4, CD8, HLA-DR, CD123 and the like, which may be combined with side scatter for gating. For example, the data points of a flow cytometry analysis may be limited to those cells that stain for CCR3, and are not high or low in granularity.

A gate in cytometry is a set of value limits (boundaries) that serve to isolate a specific group of cytometric events from a large set. Gates can be defined by discrimination analysis, or can simply be drawn around a given set of data points on a print-out and then converted to a computer-useful form. Gates can be implemented with a physical blinder. Gates may be used either to selectively gather data or to segregate data for analysis. Gates are divided mathematically into inclusive gates and exclusive gates. Inclusive gates select data that falls within the limits set, while exclusive gates select data that falls outside the limits. A live gate is a term used for a process that prevents the acquisition by the computer of non-selected data from the flow cytometer. (see, for example, Osborne, G. W. (2000) “Regions and Gates” Flow Cytometry Software Workshop: 2000, page 3).

The term “active” or “activated” may refer to having a biological or physiological effect that differs from the native biological, physiological, or wild type, state.

The term “nonactivated” may refer to a native biological, physiological, or wildtype, state.

The term “activatable” may refer to having potential to become biologically or physiologically active.

The term “normal” may refer to a standard, model, median or average of a large group. “Abnormal” may refer to a deviation of the standard, model, median or average of a large group.

The term “biological sample” may refer to a sample consisting of or containing blood, serum, plasma, lymph fluid, amniotic fluid, saliva, cerebro-spinal fluid, lacrimal fluid, mucus, urine, sputum, or sweat.

The term “contacting” may refer to a state of touching or immediate or local proximity.

The term “disease” or “disorder” may refer to an impairment of health or a condition of abnormal functioning.

The term “drug” may refer to a therapeutic agent or any substance, other than food, used in prevention, diagnosis, alleviation, treatment or cure of disease.

The term “differential label” generally may refer to a stain, dye, marker, or antibody used to characterize and/or contrast structures, components or proteins of a cell or organism.

The term “labeling” may refer to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

A kit is typically a set of reagents or devices or supplies that are packaged together in a container. The container can be divided or undivided. The reagents or devices may be combined or separate. Sufficient quantities for one or more assays may be included.

A set of assay samples are samples that have been processed to prepare for or initiate a reaction, such as activation. The samples may already have been run through the reaction and may be ready for analysis. Typically, each sample is in a separate container. Usually the samples in separate containers will be within a single or a few or more larger containers, such as test tube racks, arrays, coolers, ice bucket, ice bath, etc. Typically they set of assay sample will be within a single laboratory, a single building, or a single analytical instrument. A set of assay samples are typically identical but for one or more controlled variable elements, e.g., time of incubation.

Measurement of markers will typically be quantitative so that a rate can be determined. However, there may be embodiments in which a non-quantitative assessment may be done that will correlate with the quantitative assessment.

The term “stain” may refer to one or more dyes or pigments used to make differentiable a structure, a material, a cell, a cell component, a membrane, a granule, a nucleus, a cell surface receptor, a peptide, a microorganism, a nucleic acid, a protein or a tissue.

The term “susceptible” may refer to a member of a population at risk. The term is inclusive of a subject having a medical history of a previous allergic reaction to at least one allergen and at risk of mounting an allergic reaction to a different antigen.

The term “anaphylactic shock” may refer to a sudden, severe allergic reaction typically characterized by a sharp drop in blood pressure, urticaria, and breathing difficulties that are caused by exposure to a foreign substance after a preliminary or sensitizing exposure.

The term “expression” may refer to the use of a gene in the production of a protein, RNA, or phenotype. “Level of expression” may refer to the degree to which a particular gene produces its effect(s) in an organism.

The term “dye” (also referred to as “fluorescent moiety”) may refer to a component of a molecule which causes the molecule to be fluorescent. The component is a functional group in the molecule that absorbs energy of a specific wavelength and re-emits energy at a different, but equally specific wavelength. The amount and wavelength of the emitted energy depend on both the dye and the chemical environment of the dye.

The term “fluorescent-activated cell sorting” (also referred to as “FACS”) may refer to a method for sorting a heterogeneous mixture of biological cells into one or more containers, one cell at a time, based upon the specific light scattering and fluorescent characteristics of each cell.

The term “isolated” may refer to placing, setting apart, or obtaining a protein, molecule, substance, nucleic acid, peptide, cell or particle, in a form essentially free from contaminants or other materials with which it is commonly associated.

The term “stimulation” describes the addition of a defined amount of test allergens/antigens to a blood sample from patients with suspected allergies and subsequent incubation at controlled temperature.

The term “venipuncture” may refer to the process of obtaining intravenous access for the purpose of intravenous therapy or obtaining a sample of venous blood.

The term “whole blood” may refer to generally unprocessed or unmodified collected blood containing all of its components, such as red blood cells, white blood cells, platelets and plasma. The term “whole blood” is inclusive of any anticoagulant that may be combined with the blood upon collection.

Enhancing agent may refer to growth factors and interleukins, for example, IL-3, at a dose sufficient to increase activation markers, as described above, after 10 minutes or more of incubation of enhancing agent and test allergen with whole blood.

The above disclosure generally describes the present invention. All references disclosed are expressly incorporated by reference. A more complete understanding can be obtained by reference to the following specific examples which are provided for purposes of illustration only, and are not intended to limit the scope of the invention.

### REFERENCES

The disclosure of each reference cited is expressly incorporated.
1. Knol E F, Mul F P, Jansen H, Calafat J, Roos D. Monitoring human basophil activation via CD63 monoclonal antibody 435. J Allergy Clin Immunol. 1991;88(3 Pt 1):328-338.
2. Hausmann O V, Gentinetta T, Fux M, Ducrest S, Pichler W J, Dahinden C A. Robust expression of CCR3 as a single basophil selection marker in flow cytometry. Allergy. 2011; 66(1):85-91.
3. Hoffmann H J, Santos A F, Mayorga C, et al. The clinical utility of basophil activation testing in diagnosis and monitoring of allergic disease. Allergy. 2015; 70(11):1393-1405.
4. Santos A F, Becares N, Stephens A, Turcanu V, Lack G. The expression of CD123 can decrease with basophil activation: implications for the gating strategy of the basophil activation test. Clin Transl Allergy. 2016; 6:11.
Hennersdorf F, Florian S, Jakob A, et al. Identification of CD13, CD107a, and CD164 as novel basophil-activation markers and dissection of two response patterns in time kinetics of IgE-dependent upregulation. Cell Res. 2005; 15(5):325-335.
6. Smiljkovic M, Stanisavljevic D, Stojkovic D, et al. Apigenin-7-O-glucoside versus apigenin: Insight into the modes of anticandidal and cytotoxic actions. EXCLI J. 2017; 16:795-807.
7. Hoffmann H J, Frandsen P M, Christensen L H, Schiotz P O, Dahl R. Cultured human mast cells are heterogeneous for expression of the high-affinity IgE receptor FcepsilonRI. Int Arch Allergy Immunol. 2012; 157(3):246-250.
8. Yeung L, Hickey M J, Wright M D. The Many and Varied Roles of Tetraspanins in Immune Cell Recruitment and Migration. Front Immunol. 2018; 9:1644.
9. Shelke G V, Yin Y, Jang S C, et al. Endosomal signalling via exosome surface TGFbeta-1. J Extracell Vesicles. 2019;8(1):1650458.
10. Ebo D G, Bridts C H, Mertens C H, Hagendorens M M, Stevens W J, De Clerck L S. Analyzing histamine release by flow cytometry (HistaFlow): a novel instrument to study the degranulation patterns of basophils. J Immunol Methods. 2012; 375(1-2):30-38.
11. Hoffmann H J, Knol E F, Ferrer M, et al. Pros and Cons of Clinical Basophil Testing (BAT). Curr Allergy Asthma Rep. 2016;16(8):56.
12. Grochowy G, Hermiston M L, Kuhny M, Weiss A, Huber M. Requirement for CD45 in fine-tuning mast cell responses mediated by different ligand-receptor systems. Cell Signal. 2009; 21(8):1277-1286.
13. Xu H, Bin N R, Sugita S. Diverse exocytic pathways for mast cell mediators. Biochem Soc Trans. 2018;46(2):235-247.
14. Sander L E, Frank S P, Bolat S, et al. Vesicle associated membrane protein (VAMP)-7 and VAMP-8, but not VAMP-2 or VAMP-3, are required for activation-induced degranulation of mature human mast cells. Eur J Immunol. 2008; 38(3):855-863.
15. Klein O, Sagi-Eisenberg R. Anaphylactic Degranulation of Mast Cells: Focus on Compound Exocytosis. J Immunol Res. 2019; 2019:9542656.
16. Aranda A, Mayorga C, Ariza A, et al. In vitro evaluation of IgE-mediated hypersensitivity reactions to quinolones. Allergy. 2011; 66(2):247-254.
17. Van Gasse A L, Elst J, Bridts C H, et al. Rocuronium Hypersensitivity: Does Off-Target Occupation of the MRGPRX2 Receptor Play a Role? J Allergy Clin Immunol Pract. 2019; 7(3):998-1003.
18. Chirumbolo S, Vella A, Ortolani R, et al. Differential response of human basophil activation markers: a multi-parameter flow cytometry approach. Clin Mol Allergy. 2008; 6:12.
19. Prussin C, Metcalfe D D. 5. IgE, mast cells, basophils, and eosinophils. J Allergy Clin Immunol. 2006;117(2 Suppl Mini-Primer):5450-456.
20. Ebo D G, Bridts C H, Hagendorens M M, Aerts N E, De Clerck L S, Stevens W J. Basophil activation test by flow cytometry: present and future applications in allergology. Cytometry B Clin Cytom. 2008; 74(4):201-210.
21. Knol E F, Mul F P, Kuijpers T W, Verhoeven A J, Roos D. Intracellular events in anti-IgE nonreleasing human basophils. J Allergy Clin Immunol. 1992; 90(1):92-103.
22. MacGlashan D W, Jr. Basophil activation testing. J Allergy Clin Immunol. 2013;132(4):777-787.
23. Macglashan D, Jr., Moore G, Muchhal U. Regulation of IgE-mediated signalling in human basophils by CD32b and its role in Syk down-regulation: basic mechanisms in allergic disease. Clin Exp Allergy. 2014; 44(5):713-723.
24. MacGlashan D, Jr. Subthreshold desensitization of human basophils re-capitulates the loss of Syk and FcepsilonRI expression characterized by other methods of desensitization. Clin Exp Allergy. 2012;42(7): 1060-1070.
25. Puan K J, Andiappan A K, Lee B, et al. Systematic characterization of basophil anergy. Allergy. 2017; 72(3):373-384.
26. Santos A F, Du Toit G, O'Rourke C, et al. Biomarkers of severity and threshold of allergic reactions during oral peanut challenges. J Allergy Clin Immunol. 2020.
27. Schroeder J T, Chichester K L, Bieneman A P. Human basophils secrete IL-3: evidence of autocrine priming for phenotypic and functional responses in allergic disease. J Immunol. 2009; 182(4):2432-2438.
28. Kwok M, Lack G, Santos A F. Improved standardisation of the whole blood basophil activation test to peanut. Clin Transl Allergy. 2017;8 (Suppl 2)(26):15-16.
29. Mukai K, Gaudenzio N, Gupta S, et al. Assessing basophil activation by using flow cytometry and mass cytometry in blood stored 24 hours before analysis. J Allergy Clin Immunol. 2017; 139(3):889-899 e811.
30. M P, A N, C M, et al. Building confidence in the basophil activation test: standardization and external quality assurance—an EAACI task force Allergy. 2020.
31. Sturm G J, Kranzelbinder B, Sturm E M, Heinemann A, Groselj-Strele A, Aberer W. The basophil activation test in the diagnosis of allergy: technical issues and critical factors. Allergy. 2009; 64(9):1319-1326.
32. Patil S U, Calatroni A, Schneider M, et al. Data-driven programmatic approach to analysis of basophil activation tests. Cytometry B Clin Cytom. 2017.
33. Patil S U, Shreffler W G. Immunology in the Clinic Review Series; focus on allergies: basophils as biomarkers for assessing immune modulation. Clin Exp Immunol. 2012; 167(1):59-66.
34. MacGlashan D W, Jr. Releasability of human basophils: cellular sensitivity and maximal histamine release are independent variables. J Allergy Clin Immunol. 1993; 91(2):605-615.
35. Erdmann S M, Sachs B, Kwiecien R, Moll-Slodowy S, Sauer I, Merk H F. The basophil activation test in wasp venom allergy: sensitivity, specificity and monitoring specific immunotherapy. Allergy. 2004;59(10): 1102-1109.
36. Santos A F, Du Toit G, Douiri A, et al. Distinct parameters of the basophil activation test reflect the severity and threshold of allergic reactions to peanut. J Allergy Clin Immunol. 2015; 135(1):179-186.
37. Johansson S G, Nopp A, van Hage M, et al. Passive IgE-sensitization by blood transfusion. Allergy. 2005;60(9): 1192-1199.
38. Dahlen B, Nopp A, Johansson S G, Eduards M, Skedinger M, Adedoyin J. Basophil allergen threshold sensitivity, CD-sens, is a measure of allergen sensitivity in asthma. Clin Exp Allergy. 2011;41(8): 1091-1097.
39. Nopp A, Cardell L O, Johansson S G. CD-sens can be a reliable and easy-to-use complement in the diagnosis of allergic rhinitis. Int Arch Allergy Immunol. 2013; 161(1):87-90.
40. Glaumann S, Nopp A, Johansson S G, Rudengren M, Bones M P, Nilsson C. Basophil allergen threshold sensitivity, CD-sens, IgE-sensitization and DBPCFC in peanut-sensitized children. Allergy. 2012; 67(2):242-247.
41. Nilsson N, Nilsson C, Hedlin G, Johansson S G, Bones M P, Nopp A. Combining analyses of basophil allergen threshold sensitivity, CD-sens, and IgE antibodies to hydrolyzed wheat, omega-5 gliadin and timothy grass enhances the prediction of wheat challenge outcome. Int Arch Allergy Immunol. 2013; 162(1):50-57.
42. Santos A F, Douiri A, Becares N, et al. Basophil activation test discriminates between allergy and tolerance in peanut-sensitized children. J Allergy Clin Immunol. 2014; 134(3):645-652.
43. Nopp A, Cardell L O, Johansson S G, Oman H. CD-sens: a biological measure of immunological changes stimulated by ASIT. Allergy. 2009; 64(5):811-814.
44. Schmid J M, Wurtzen P A, Dahl R, Hoffmann H J. Early improvement in basophil sensitivity predicts symptom relief with grass pollen immunotherapy. J Allergy Clin Immunol. 2014; 134(3):741-744 e745.
45. Kosnik M, Silar M, Bajrovic N, Music E, Korosec P. High sensitivity of basophils predicts side-effects in venom immunotherapy. Allergy. 2005; 60(11):1401-1406.
46. Lalek N, Kosnik M, Silar M, Korosec P. Immunoglobulin G-dependent changes in basophil allergen threshold sensitivity during birch pollen immunotherapy. Clin Exp Allergy. 2010; 40(8):1186-1193.
47. Schmid J M, Wurtzen P A, Siddhuraj P, et al. Basophil sensitivity reflects long-term clinical outcome of subcutaneous immunotherapy in grass pollen-allergic patients. Allergy. 2020.
48. Nopp A, Johansson S G, Ankerst J, et al. Basophil allergen threshold sensitivity: a useful approach to anti-IgE treatment efficacy evaluation. Allergy. 2006; 61(3):298-302.
49. Nopp A, Johansson S G, Ankerst J, Palmqvist M, Oman H. CD-sens and clinical changes during withdrawal of Xolair after 6 years of treatment. Allergy. 2007; 62(10):1175-1181.
50. Nopp A, Johansson S G, Adedoyin J, Ankerst J, Palmqvist M, Oman H. After 6 years with Xolair; a 3-year withdrawal follow-up. Allergy. 2010; 65(1):56-60.
51. Konradsen J R, Nordlund B, Nilsson O B, et al. High basophil allergen sensitivity (CD-sens) is associated with severe allergic asthma in children. Pediatr Allergy Immunol. 2012; 23(4):376-384.
52. Zidarn M, Kosnik M, Silar M, Grahek A, Korosec P. Rhinitis symptoms caused by grass pollen are associated with elevated basophile allergen sensitivity and a larger grass-specific immunoglobulin E fraction. Clin Exp Allergy. 2012; 42(1):49-57.
53. Zidarn M, Kosnik M, Silar M, Bajrovic N, Korosec P. Sustained effect of grass pollen subcutaneous immunotherapy on suppression of allergen-specific basophil response; a real-life, nonrandomized controlled study. Allergy. 2015; 70(5):547-555.
54. Brandstrom J, Nopp A, Johansson S G, et al. Basophil allergen threshold sensitivity and component-resolved diagnostics improve hazelnut allergy diagnosis. Clin Exp Allergy. 2015; 45(9):1412-1418.
55. Santos A F, Shreffler W G. Road map for the clinical application of the basophil activation test in food allergy. Clin Exp Allergy. 2017; 47(9):1115-1124.
56. Hemmings O, Kwok M, McKendry R, Santos A F. Basophil Activation Test: Old and New Applications in Allergy. Curr Allergy Asthma Rep. 2018;18(12):77.
57. Santos A F, Du Toit G, O'Rourke C, et al. Identifying allergic children with severe adverse events during oral peanut challenges in the LEAP studies by assessing basophil activation. Allergy. 2019;74(S 106):73.
58. Santos A F, Lack G. Basophil activation test: food challenge in a test tube or specialist research tool? Clin Transl Allergy. 2016; 6:10.
59. Eberlein B, Krischan L, Darsow U, Ollert M, Ring J. Double positivity to bee and wasp venom: improved diagnostic procedure by recombinant allergen-based IgE testing and basophil activation test including data about cross-reactive carbohydrate determinants. J Allergy Clin Immunol. 2012; 130(1):155-161.
60. Arzt L, Bokanovic D, Schrautzer C, et al. Immunological differences between insect venom-allergic patients with and without immunotherapy and asymptomatically sensitized subjects. Allergy. 2018;73 (6): 1223-1231.
61. Wanich N, Nowak-Wegrzyn A, Sampson H A, Shreffler W G. Allergen-specific basophil suppression associated with clinical tolerance in patients with milk allergy. J Allergy Clin Immunol. 2009; 123(4):789-794 e720.
62. Berin M C, Grishin A, Masilamani M, et al. Egg-specific IgE and basophil activation but not egg-specific T-cell counts correlate with phenotypes of clinical egg allergy. J Allergy Clin Immunol. 2018; 142(1):149-158 e148.
63. Song Y, Wang J, Leung N, et al. Correlations between basophil activation, allergen-specific IgE with outcome and severity of oral food challenges. Ann Allergy Asthma Immunol. 2015; 114(4):319-326.
64. Rubio A, Vivinus-Nebot M, Bourrier T, Saggio B, Albertini M, Bernard A. Benefit of the basophil activation test in deciding when to reintroduce cow's milk in allergic children. Allergy. 2011; 66(1):92-100.
65. Chinthrajah R S, Purington N, Andorf S, et al. Development of a tool predicting severity of allergic reaction during peanut challenge. Ann Allergy Asthma Immunol. 2018; 121(1):69-76 e62.
66. Reier-Nilsen T, Michelsen M M, Lodrup Carlsen K C, et al. Predicting reactivity threshold in children with anaphylaxis to peanut. Clin Exp Allergy. 2018; 48(4):415-423.
67. Burks A W, Jones S M, Wood R A, et al. Oral immunotherapy for treatment of egg allergy in children. N Engl J Med. 2012; 367(3):233-243.
68. Vickery B P, Scurlock A M, Kulis M, et al. Sustained unresponsiveness to peanut in subjects who have completed peanut oral immunotherapy. J Allergy Clin Immunol. 2014; 133(2):468-475.
69. Shamji M H, Layhadi J A, Scadding G W, et al. Basophil expression of diamine oxidase: a novel biomarker of allergen immunotherapy response. J Allergy Clin Immunol. 2015; 135(4):913-921 e919.
70. Thyagarajan A, Jones S M, Calatroni A, et al. Evidence of pathway-specific basophil anergy induced by peanut oral immunotherapy in peanut-allergic children. Clin Exp Allergy. 2012; 42(8):1197-1205.
71. Gorelik M, Narisety S D, Guerrerio A L, et al. Suppression of the immunologic response to peanut during immunotherapy is often transient. J Allergy Clin Immunol. 2015; 135(5):1283-1292.
72. Hoffmann H J, Valovirta E, Pfaar O, et al. Novel approaches and perspectives in allergen immunotherapy. Allergy. 2017;72(7): 1022-1034.
73. Santos A F, James L K, Kwok M, et al. Peanut oral immunotherapy induces blocking antibodies but does not change functional characteristics of peanut-specific IgE. J Allergy Clin Immunol. 2019.
74. Patil S U, Steinbrecher J, Calatroni A, et al. Early decrease in basophil sensitivity to Ara h 2 precedes sustained unresponsiveness after peanut oral immunotherapy. J Allergy Clin Immunol. 2019; 144(5):1310-1319 e1314.
75. Santos A F, James L K, Bahnson H T, et al. IgG4 inhibits peanut-induced basophil and mast cell activation in peanut-tolerant children sensitized to peanut major allergens. J Allergy Clin Immunol. 2015; 135(5):1249-1256.
76. McKendry R T, Kwok M, Hemmings O, James L K, AF S. Basophil and mast cell responses to food allergens in sensitised but tolerant patients are not mediated via the FcgRII and FcgRII receptors. Allergy. 2019;74(S106):97.
77. MacGlashan D W, Jr., Saini S S. Syk expression and IgE-mediated histamine release in basophils as biomarkers for predicting the clinical efficacy of omalizumab. J Allergy Clin Immunol. 2017; 139(5):1680-1682 e1610.
78. MacGlashan D W, Jr., Savage J H, Wood R A, Saini S S. Suppression of the basophil response to allergen during treatment with omalizumab is dependent on 2 competing factors. J Allergy Clin Immunol. 2012; 130(5):1130-1135 e1135.
79. Savage J H, Courneya J P, Sterba P M, Macglashan D W, Saini S S, Wood R A. Kinetics of mast cell, basophil, and oral food challenge responses in omalizumab-treated adults with peanut allergy. J Allergy Clin Immunol. 2012; 130(5): 1123-1129 e1122.
80. Macglashan D W, Jr., Saini S S. Omalizumab increases the intrinsic sensitivity of human basophils to IgE-mediated stimulation. J Allergy Clin Immunol. 2013; 132(4):906-911 e901-904.
81. Eckman J A, Sterba P M, Kelly D, et al. Effects of omalizumab on basophil and mast cell responses using an intranasal cat allergen challenge. J Allergy Clin Immunol. 2010; 125(4):889-895 e887.
82. Johansson S G, Nopp A, Oman H, et al. The size of the disease relevant IgE antibody fraction in relation to ‘total-IgE’ predicts the efficacy of anti-IgE (Xolair) treatment. Allergy. 2009; 64(10):1472-1477.
83. Patil S U, Calatroni A, Schneider M, et al. Data-driven programmatic approach to analysis of basophil activation tests. Cytometry B Clin Cytom. 2018; 94(4):667-673.
84. Depince-Berger A E, Sidi-Yahya K, Jeraiby M, Lambert C. Basophil activation test: Implementation and standardization between systems and between instruments. Cytometry A. 2017; 91(3):261-269.
85. Uyttebroek A P, Sabato V, Faber M A, et al. Basophil activation tests: time for a reconsideration. Expert Rev Clin Immunol. 2014;10(10): 1325-1335.
86. Ryherd M, Plassmeyer M, Alexander C, et al. Improved panels for clinical immune phenotyping: Utilization of the violet laser. Cytometry B Clin Cytom. 2018; 94(5):671-679.
87. Mukai K, Gaudenzio N, Gupta S, et al. Assessing basophil activation by using flow cytometry and mass cytometry in blood stored 24 hours before analysis. J Allergy Clin Immunol. 2017; 139(3):889-899.e811.
88. Davis B H, Wood B, Oldaker T, Barnett D. Validation of cell-based fluorescence assays: practice guidelines from the ICSH and ICCS—part I—rationale and aims. Cytometry B Clin Cytom. 2013; 84(5):282-285.
89. Davis B H, Dasgupta A, Kussick S, Han J Y, Estrellado A. Validation of cell-based fluorescence assays: practice guidelines from the ICSH and ICCS—part II—preanalytical issues. Cytometry B Clin Cytom. 2013; 84(5):286-290.
90. Barnett D, Louzao R, Gambell P, De J, Oldaker T, Hanson C A. Validation of cell-based fluorescence assays: practice guidelines from the ICSH and ICCS—part IV—postanalytic considerations. Cytometry B Clin Cytom. 2013; 84(5):309-314.
91. Tsai M, Mukai K, Chinthrajah R S, Nadeau K C, Galli S J. Sustained successful peanut oral immunotherapy associated with low basophil activation and peanut-specific IgE. J Allergy Clin Immunol. 2020; 145(3):885-896 e886.
92. Santos A F, Couto-Francisco N, Becares N, Kwok M, Bahnson H T, Lack G. A novel human mast cell activation test for peanut allergy. J Allergy Clin Immunol. 2018; 142(2):689-691 e689.
93. Kleine-Tebbe J, Erdmann S, Knol E F, MacGlashan D W, Jr., Poulsen L K, Gibbs B F. Diagnostic tests based on human basophils: potentials, pitfalls and perspectives. Int Arch Allergy Immunol. 2006; 141(1):79-90.
94. de Weck A L, Sanz M L, Gamboa P M, et al. Diagnostic tests based on human basophils: more potentials and perspectives than pitfalls. Int Arch Allergy Immunol. 2008; 146(3):177-189.
95. Wojtalewicz N, Goseberg S, Kabrodt K, Schellenberg I. Six years of INSTAND e. V. sIgE proficiency testing: An evaluation of in vitro allergy diagnostics. Allergo J Int. 2017;26(2):43-52.
96. Korosec P, Turner P J, Silar M, et al. Basophils, high-affinity IgE receptors, and CCL2 in human anaphylaxis. J Allergy Clin Immunol. 2017; 140(3):750-758 e715.
97. Ocmant A, Mulier S, Hanssens L, et al. Basophil activation tests for the diagnosis of food allergy in children. Clin Exp Allergy. 2009; 39(8):1234-1245.
98. Eberlein B, Leon Suarez I, Darsow U, Rueff F, Behrendt H, Ring J. A new basophil activation test using CD63 and CCR3 in allergy to antibiotics. Clin Exp Allergy. 2010; 40(3):411-418.
99. Leysen J, Bridts C H, De Clerck L S, Ebo D G. Rocuronium-induced anaphylaxis is probably not mitigated by sugammadex: evidence from an in vitro experiment. Anaesthesia. 2011;66(6):526-527.
100. Sturm G J, Bohm E, Trummer M, Weiglhofer I, Heinemann A, Aberer W. The CD63 basophil activation test in Hymenoptera venom allergy: a prospective study. Allergy. 2004; 59(10):1110-1117.
101. http://cls.syr.edu/mathtuneup/graphb/unit8/Unit8a.html

We claim:

1. A method to prepare samples to aid in determining likelihood of a subject having an allergic reaction against an allergen of interest, the method comprising:
stimulating in vitro basophil cells in a first blood sample of the subject by contacting the basophil cells with a selected concentration of the allergen of interest for a first period of time;
stimulating basophil cells in vitro in a second blood sample of the subject by contacting the basophil cells with the selected concentration of the allergen of interest for a second period of time; and
subjecting the first and second blood samples to an analysis of basophil cell activation and determining the rate of change between the first and second time periods.

2. The method of claim 1 wherein the first and second blood samples are formed by withdrawal and quenching of two aliquots from a common stimulation reaction.

3. The method of claim 1 wherein basophilic cells in additional blood samples are stimulated for additional periods of time.

4. The method of claim 1 wherein basophil cells are stimulated for between three and ten time periods to form from three to ten blood samples.

5. The method of claim 1 wherein the analysis is by flow cytometry.

6. The method of claim 1 wherein the allergen is a food allergen.

7. The method of claim 1 wherein the allergen is an environmental allergen.

8. The method of claim 1 wherein the allergen is peanut protein or peanut protein component.

9. The method of claim 1 wherein the subject has previously been subjected to desensitization immunization therapy with the allergen.

10. A method of treating a subject who is prone to having an allergic reaction against an allergen of interest, the method comprising:
stimulating in vitro basophil cells in a first blood sample of the subject by contacting the basophil cells with a selected concentration of the allergen of interest for a first period of time;
stimulating basophil cells in vitro in a second blood sample of the subject by contacting the basophil cells with the selected concentration of the allergen of interest for a second period of time;
measuring one or more markers of basophil activation in the first and second blood samples;
determining a rate of basophil activation by determining a change in amount of the one or more markers between the first and second blood samples; and
treating the subject with desensitizing immunotherapy toward the allergen of interest when the rate is determined to be significantly above negative control values.

11. The method of claim 10 wherein the first and second blood samples are formed by withdrawal and quenching of two aliquots from a common stimulation reaction.

12. The method of claim 10 wherein basophilic cells in additional blood samples of the subject are stimulated for additional periods of time.

13. The method of claim 12 wherein basophil cells are stimulated for between three and ten time periods.

14. The method of claim 10 wherein the one or more markers are intracellular phosphorylated proteins.

15. The method of claim 10 wherein the one or more markers are cell surface markers.

16. The method of claim 10 wherein the one or more markers comprise at least one cell surface marker selected from the group consisting of CD203c, MRGPRX2 and CD63.

17. The method of claim 14 wherein the one or more intracellular phosphorylated proteins comprise at least one selected from the group consisting of phospho-Lyn, phospho-Fyn, phospho AKT, phospho S6, phospho-IgE-receptor, and combinations thereof.

18. The method of claim 10 wherein at least one cell surface marker and once intracellular phosphorylated protein are measured in the first and second blood sample.

19. The method of claim 10 wherein the step of measuring employs one or more antibodies which specifically bind to the one or more markers of basophil activation.

20. The method of claim 10 wherein the step of measuring employs flow cytometry.

21. The method of claim 10 wherein the allergen of interest is a food allergen.

22. The method of claim 10 wherein the allergen of interest is an environmental allergen.

23. The method of claim 10 wherein the allergen of interest is peanut protein.

24. The method of claim 10 wherein the subject has previously been subjected to desensitization immunization therapy.

25. A set of assay samples to aid in determining likelihood of a subject having an allergic reaction against an allergen of interest, the set comprising:
a plurality of assay samples comprising at least a first and a second blood sample formed by stimulating in vitro basophil cells in a first blood sample of the subject by contacting the basophil cells with a selected concentration of the allergen of interest for a first period of time and by stimulating basophil cells in vitro in a second blood sample of the subject by contacting the basophil cells with the selected concentration of the allergen of interest for a second period of time;
wherein the plurality of assay samples are suitable for measuring one or more markers of basophil activation in the blood samples whereby a rate of basophil activation can be determined by determining a change in amount of the one or more markers between any of the blood samples.

26. The set of claim 25 wherein the first and second blood samples are formed by withdrawal and quenching of two aliquots from a common stimulation reaction.

27. The set of claim 25 wherein basophilic cells in additional blood samples are stimulated for additional periods of time.

28. The set of claim 25 wherein basophil cells in three to ten assay samples are stimulated for three to ten time periods.

29. The set of claim 25 wherein the allergen of interest is a food allergen.

30. The set of claim 25 wherein the allergen of interest is an environmental allergen.

31. The set of claim 25 wherein the allergen of interest is peanut protein.

32. The set of claim 25 wherein the subject has previously been subjected to desensitization immunization therapy.

33. A kit to aid in determining likelihood of a subject having an allergic reaction against an allergen of interest, the method comprising:
a plurality of tubes for holding at least a first and a second blood sample of the subject in which basophil cells will be stimulated by contacting the basophil cells with a selected concentration of the allergen of interest;
the allergen of interest at a predetermined amount in separate packages to be added to the at least first and second blood samples; and
a quenching agent for stopping the stimulation of basophil cells at a predetermined amount in separate packages to be added to the at least first and second blood samples.

34. The kit of claim 33 further comprising:
a timer for measuring basophil cell stimulation times for the at least first and second blood samples.

35. The kit of claim 33 further comprising:
a plurality of timers for predetermined basophil cell stimulation times for the at least first and second blood samples.

36. The kit of claim 33 further comprising:
at least one antibody specific for a basophil activation marker.

37. The kit of claim 33 further comprising:
at least one antibody specific for a basophil activation marker, wherein the antibody is attached to a fluorescent moiety.

38. The kit of claim 33 further comprising:
a plurality of antibodies, wherein each antibody is specific for a basophil activation marker, wherein each antibody is attached to a different fluorescent moiety.

39. A method to aid in determining likelihood of a subject having an allergic reaction against an allergen of interest, the method comprising:
stimulating in vitro basophil cells in a first blood sample of the subject by contacting the basophil cells with a selected concentration of the allergen of interest for a first period of time;
stimulating basophil cells in vitro in a second blood sample of the subject by contacting the basophil cells with the selected concentration of the allergen of interest for a second period of time;
measuring one or more markers of basophil activation in the first and second blood samples; and
determining a rate of basophil activation by determining a change in amount of the one or more markers between the first and second blood samples.

40. The method of claim 39 wherein the first and second blood samples are formed by withdrawal and quenching of two aliquots from a common stimulation reaction.

41. The method of claim 39 wherein basophilic cells in additional blood samples are stimulated for additional periods of time.

42. The method of claim 39 wherein basophil cells in three to ten blood samples are stimulated for three to ten time periods, respectively.

43. The method of claim 39 wherein the one or more markers are intracellular phosphorylated proteins.

44. The method of claim 39 wherein the one or more markers are cell surface markers.

45. The method of claim 39 wherein the one or more markers comprise at least one cell surface marker selected from the group consisting of CD203c, MRGPRX2 and CD63.

46. The method of claim 43 wherein the one or more intracellular phosphorylated proteins comprise at least one selected from the group consisting of phospho-Lyn, phospho-Fyn, phospho-IgE-receptor, and combinations thereof.

47. The method of claim 39 wherein antibodies which specifically bind to at least one cell surface marker and at least one intracellular phosphorylated protein are contacted with the first and second blood sample.

48. The method of claim 39 wherein the step of measuring employs one or more antibodies which specifically bind to the one or more markers of basophil activation.

49. The method of claim 39 wherein the step of measuring employs flow cytometry.

50. The method of claim 39 wherein the allergen of interest is a food allergen.

51. The method of claim 39 wherein the allergen of interest is an environmental allergen.

52. The method of claim 39 wherein the allergen of interest is peanut protein.

53. The method of claim 39 wherein the subject has previously been subjected to desensitization immunization therapy.

54. The method of claim 39 wherein the subject is recommended for desensitization therapy on the basis of the determined rate of basophil activation.

55. The method of claim 8 wherein the allergen is peanut protein component and the peanut protein component is selected from the group consisting of: Arah1, Arah2, Arah6, Arah7, AraH8 and Arah10.
